(19) United States
(12) Patent Application Publication
(10) Pub. No.: US 2023/0314345 A1
(43) Pub. Date: Oct. 5, 2023

(54) GAS SENSOR DEVICES, METHODS FOR PRODUCING SAME, AND METHODS FOR GENERATING ABSORPTION SPECTRA OF GASES

(71) Applicant: Infineon Technologies AG, Neubiberg (DE)

(72) Inventors: Bernhard RIEDER, Regensburg (DE); Rainer Markus SCHALLER, Aichen (DE); Maciej WOJNOWSKI, Muenchen (DE)

(21) Appl. No.: 18/184,818

(22) Filed: Mar. 16, 2023

(30) Foreign Application Priority Data
Mar. 29, 2022 (DE) 102022107320.3

(51) Int. Cl. G01N 22/00

(52) U.S. Cl. CPC G01N 22/00

(57) ABSTRACT

A gas sensor device contains a cavity delimited by an electrically conductive material and having gas-permeable openings and reflective surfaces, and also a radio-frequency component, including a radio-frequency chip and at least one radio-frequency antenna configured to emit radio-frequency signals into the cavity and to receive radio-frequency signals from the cavity.

### CROSS REFERENCE TO RELATED APPLICATION

This application claims priority to German Patent Application No. 102022107320.3 filed on Mar. 29, 2022, the content of which is incorporated by reference herein in its entirety.

### TECHNICAL FIELD

The present disclosure relates to gas sensor devices and methods for producing such gas sensor devices. Furthermore, the present disclosure relates to methods for generating absorption spectra of gases.

### BACKGROUND

Gas sensors can be used in a multiplicity of technical applications. In one example, harmful or dangerous components of the ambient air can be detected with the aid of a gas sensor. In a further example, a respiratory gas analysis can be carried out using a gas sensor and lung diseases or diseases of other organs can be deduced as a result. Many of the gas sensors used for the purposes mentioned may have complex designs and/or unwieldy dimensions. Manufacturers and developers of gas sensor devices are constantly endeavoring to improve their products. In this context, it may be desirable to provide cost-effective gas sensors having compact dimensions. Furthermore, it may be desirable to improve methods for producing and for using such gas sensors.

### SUMMARY

Various aspects relate to a gas sensor device. The gas sensor device includes a cavity delimited by an electrically conductive material and having gas-permeable openings and reflective surfaces. The gas sensor device furthermore includes a radio-frequency component, including a radio-frequency chip and at least one radio-frequency antenna configured to emit radio-frequency signals into the cavity and to receive radio-frequency signals from the cavity.

Various aspects relate to a method for generating an absorption spectrum of a gas. The method includes enabling a gas to penetrate into a cavity delimited by an electrically conductive material and having reflective surfaces by way of gas-permeable openings of the cavity. The method furthermore includes emitting radio-frequency signals into the cavity, wherein the radio-frequency signals are in a frequency range which includes at least one absorption frequency of the gas. The method furthermore includes receiving radio-frequency signals from the cavity, wherein the received radio-frequency signals have passed through the gas in the cavity. The method furthermore includes generating the absorption spectrum of the gas based on the received radio-frequency signals.

Various aspects relate to a method for producing a gas sensor device. The method includes producing a cavity delimited by an electrically conductive material and having gas-permeable openings and reflective surfaces. The method furthermore includes producing a radio-frequency component, including a radio-frequency chip and at least one radio-frequency antenna configured to emit radio-frequency signals into the cavity and to receive radio-frequency signals from the cavity.

### BRIEF DESCRIPTION OF THE DRAWINGS

Devices and methods in accordance with the disclosure are explained in greater detail below with reference to drawings. The elements shown in the drawings are not necessarily rendered in a manner true to scale relative to one another. Identical reference signs may designate identical components.

FIG. 1 schematically shows a cross-sectional side view of a gas sensor device 100 in accordance with the disclosure.

FIG. 2 schematically shows a cross-sectional side view of a gas sensor device 200 in accordance with the disclosure.

FIG. 3 schematically shows a cross-sectional side view of a gas sensor device 300 in accordance with the disclosure.

FIG. 4 schematically shows a cross-sectional side view of a gas sensor device 400 in accordance with the disclosure.

FIG. 5 schematically shows a cross-sectional side view of a gas sensor device 500 in accordance with the disclosure.

FIG. 6 schematically shows a cross-sectional side view of a gas sensor device 600 in accordance with the disclosure.

FIGS. 7A and 7B schematically show a top view and a bottom view of a radio-frequency component that can be contained in a gas sensor device in accordance with the disclosure.

FIG. 8 shows an example frequency profile of a chirp signal.

FIG. 9 shows a flow diagram of a method in accordance with the disclosure for generating an absorption spectrum of a gas.

FIG. 10 shows a flow diagram of a method in accordance with the disclosure for producing a gas sensor device.

### DETAILED DESCRIPTION

In the following detailed description, reference is made to the accompanying drawings, which show for illustration purposes specific aspects and implementations in which the disclosure can be implemented in practice. In this context, direction terms such as, for example, “at the top”, “at the bottom”, “at the front”, “at the back”, etc. can be used with respect to the orientation of the figures described. Since the components of the implementations described can be positioned in different orientations, the direction terms can be used for illustration purposes and are not restrictive in any way whatsoever. Other aspects can be used and structural or logical changes can be made, without departing from the concept of the present disclosure. That is to say that the following detailed description should not be understood in a restrictive sense.

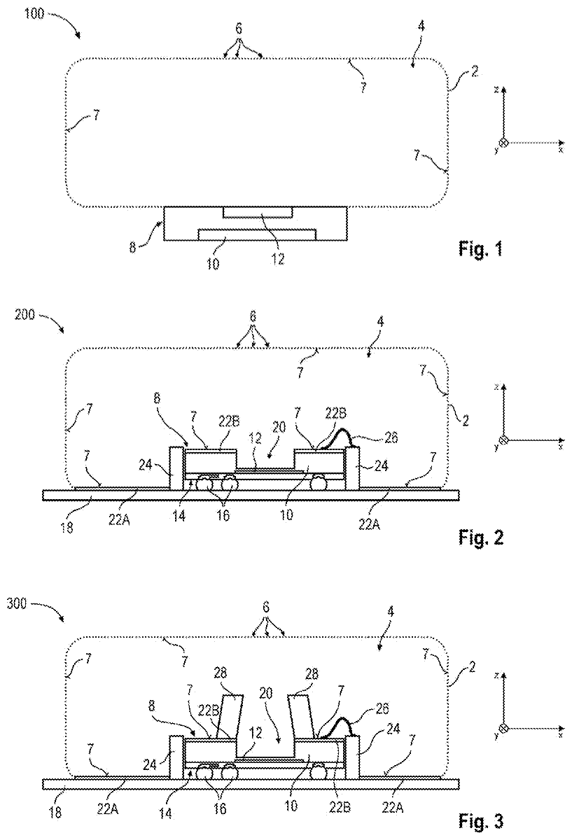

The gas sensor device 100 in FIG. 1 is illustrated in a general way in order to qualitatively describe aspects of the disclosure. The gas sensor device 100 can have further aspects that are not shown in FIG. 1 for the sake of simplicity. By way of example, the gas sensor device 100 can be extended by any aspects that are described in conjunction with other devices or methods in accordance with the disclosure.

The gas sensor device 100 can comprise a cavity 4 delimited by an electrically conductive material 2 and having gas-permeable openings 6 and reflective surfaces 7. Furthermore, the gas sensor device 100 can comprise a radio-frequency component 8 having a radio-frequency chip 10 and at least one radio-frequency antenna 12. The at least one radio-frequency antenna 12 can be configured to emit radio-frequency signals into the cavity 4 and to receive radio-frequency signals from the cavity 4. The following description reveals, inter alia, that the electrically conductive material 2 delimiting the cavity 4 and the radio-frequency component 8 can be embodied in different ways and do not have to be restricted to a specific implementation.

The gas sensor device 100 can be configured to generate an absorption spectrum of a gas situated in the cavity 4. In this context, the gas sensor device 100 need not necessarily be restricted to a specific technical application. In one example, the gas sensor device 100 can be configured to detect harmful or dangerous components of the ambient air based on the absorption spectrum generated. In a further example, using the gas sensor device 100, a respiratory gas analysis can be carried out and gases which allow lung diseases or diseases of other organs to be deduced can be detected based on a generated absorption spectrum of the respiratory air.

A gas to be examined can firstly pass into the cavity 4 by way of the gas-permeable openings 6. If the cavity 4 is filled with the gas, the at least one radio-frequency antenna 12 can emit radio-frequency signals into the cavity 4 in a frequency range which can comprise one or more absorption frequencies of the gas. The radio-frequency signals can be for example chirp signals such as are described and shown by way of example in FIG. 8. The radio-frequency signals emitted into the cavity 4 can be reflected multiple times at the inner surfaces 7 of the electrically conductive material 2 and can thus pass through the gas multiple times. In this case, electromagnetic radiation having such frequencies which correspond to the absorption frequencies of the gas can be absorbed by the gas. Accordingly, the at least one radio-frequency antenna 12 can receive from the cavity 4 radio-frequency signals having reduced intensities at the absorption frequencies of the gas.

The signals received by the at least one radio-frequency antenna 12 can be forwarded to a component configured to process the received signals and to provide an absorption spectrum of the gas to be examined based on the processed signals. In one example, this processing component can be the radio-frequency chip 10 or an integrated circuit therein. In a further example, the gas sensor device 100 can comprise some other integrated circuit situated outside or within the radio-frequency component 8 for the processing of the received signals. One or more gas species present and also the concentration(s) thereof can be deduced based on the absorption spectrum generated.

The at least one radio-frequency antenna 12 can comprise one or more transmitting and receiving antennas and can be electrically coupled to the radio-frequency chip 10. As a result, the radio-frequency chip 10 can provide data to be emitted to a transmitting antenna, and radio-frequency signals received by a receiving antenna can be forwarded to the radio-frequency chip 10 for the purpose of processing. The radio-frequency chip 10 can comprise or correspond to a monolithic microwave integrated circuit (MMIC), in particular. The radio-frequency chip 10 can operate in different frequency ranges. Accordingly, the at least one radio-frequency antenna 12 electrically coupled to the radio-frequency chip 10 can be configured to emit and/or to receive signals having frequencies in these frequency ranges. In general, a frequency of the radio-frequency signals emitted into the cavity 4 by the at least one radio-frequency antenna 12 can be in a range of approximately 100 GHz to approximately 1 THz. More specifically, a frequency of the emitted radio-frequency signals can be in one or more subranges of the aforementioned frequency range. These subranges can contain in particular one or more absorption frequencies of a gas to be examined.

The electrically conductive material 2 forming or delimiting the cavity 4 can form a Faraday cage around the cavity 4. A Faraday cage can be described as an enclosure that is closed substantially on all sides and is composed of an electrically conductive material configured to act as an electrical shield. The cavity 4 can directly adjoin the electrically conductive material 2, that is to say that it is possible for no electrically insulating material to be arranged between the inner side of the electrically conductive material 2 and the cavity 4. The electrically conductive material 2 can be chosen as desired and can be produced in particular from a metal or a metal alloy. In FIG. 1, the electrically conductive material 2 can be embodied by way of example in the form of a metal cover having openings 6 formed therein. In order to provide an enclosure that is closed on all sides and is composed of electrically conductive material, an electrically conductive material can additionally be arranged on the top side of the radio-frequency component 8, which material can terminate flush with the metal cover.

Dimensions (in particular maximum dimensions) of the openings 6 can be configured to the effect that the cavity 4 forms a cavity resonator for radio-frequency signals emitted into the cavity 4. A cavity resonator can be described as a hollow, closed electrical conductor which can contain electromagnetic waves (in particular radio-frequency waves) that are reflected back and forth between the walls of the cavity. The cavity 4 or the electrically conductive material 2 can be configured to be substantially non-transmissive to radio-frequency signals emitted into the cavity 4 by the radio-frequency antenna 12 and to confine or store the signals by way of reflections at the inner surfaces 7 of the electrically conductive material 2. This can hold true both for radio-frequency signals radiated in with discrete frequency values and for radio-frequency signals radiated in with a continuous frequency spectrum.

Of course, in practice the cavity resonator need not necessarily provide one hundred percent non-transmissivity, since power losses should always be expected during real operation of the gas sensor device 100. The transmissivity or non-transmissivity of the cavity resonator can be described by way of a quality factor of the cavity or cavity resonator. The quality factor (or Q-factor) of a cavity resonator can correspond to a ratio between the stored energy of the cavity and the power losses in the cavity. Power losses that occur in the cavity 4 and the quality factor can each be divided into two portions here. A first portion of the power losses can be caused by dissipation in a dielectric material filling the cavity 4. In the present case, power losses can be caused in particular by a gas situated in the cavity 4. An associated quality factor (Q_{d}) can be referred to as a dielectric quality factor. A second portion of the power losses can be caused by resistance losses in the metallic part of the cavity resonator. In this context, in particular, surface currents induced on the inner surfaces 7 of the cavity 4 can cause power losses. An associated quality factor (Q_{c}) can be referred to as a conductive quality factor.

The cavity or cavity resonator 4 can have a high quality factor on account of a low electrical resistance of the electrically conductive material 2. In the present case, a quality factor of the cavity resonator (in particular the conductive quality factor Q_{c}) can be greater than approximately 1·10³, or greater than approximately 5·10³, or greater than approximately 10·10³, or greater than approximately 15·10³, or greater than approximately 20·10³. In one example, an increased value of the quality factor can be provided by virtue of the electrically conductive material 2 having a coating arranged on the inner surface 7 of the cavity 4. The coating can be produced from an arbitrary highly conductive material. By way of example, the coating can contain a noble metal or a semi-noble metal, in particular one or more out of gold, silver, copper. In a further example, an increased value of the quality factor can be provided by virtue of the electrically conductive material 2 having a layer stack arranged on the inner surface 7 of the cavity 4. The layer stack can comprise at least one ferromagnetic layer (e.g., nickel) and at least one electrically conductive layer (e.g., copper). The ferromagnetic and electrically conductive layers can be stacked in particular alternately one above another. Layer stacks having such properties can be referred to as meta-conductors.

In order to prevent radio-frequency signals radiated into the cavity 4 from leaving the cavity 4 through the openings 6, dimensions of the openings 6 can be chosen to be correspondingly small. For this purpose, an (in particular maximum) dimension of the openings 6 can be smaller than approximately a wavelength λ of the radio-frequency signals emitted into the cavity 4, or smaller than approximately λ/2, or smaller than approximately λ/4, or smaller than approximately λ/6, or smaller than approximately λ/8, or smaller than approximately λ/10. At the same time, the dimensions of the openings 6 can be chosen in such a way that a gas to be examined can penetrate into the cavity 4 by way of the openings 6. In other words, the openings 6 can be permeable to the gas, but non-transmissive to the radio-frequency signals. In FIG. 1, by way of example, the openings 6 can be distributed over the entire surface of the electrically conductive material 2. Alternatively, in further examples, the openings 6 can be arranged just at one or a plurality of locations, while the rest of the surface of the electrically conductive material 2 can be closed. The openings 6 can have one or more arbitrary shapes, for example at least one out of round, circular, elliptic, rectangular, square, polygonal, slotted, bar-shaped, etc.

A dimension of the cavity 4 can be dependent on the wavelength λ of the radio-frequency signals emitted into the cavity 4 by the at least one radio-frequency antenna 12. A maximum dimension of the cavity 4 in each spatial direction can be in a range of from approximately 2 times the wavelength λ up to approximately 50 times the wavelength λ, or in a range of from approximately 2 times the wavelength λ up to approximately 40 times the wavelength λ, or in a range of from approximately 2 times the wavelength λ up to approximately 30 times the wavelength λ, or in a range of from approximately 2 times the wavelength λ up to approximately 20 times the wavelength λ, or in a range of from approximately 2 times the wavelength λ up to approximately 10 times the wavelength λ. Taking into account the frequency ranges specified above, a dimension of the cavity 4 can thus amount to one or more centimeters, for example. In comparison with conventional arrangements, the gas sensor device 100 can have more compact dimensions. In the side view in FIG. 1, by way of example, the cavity 4 can have a substantially rectangular shape, that is to say that the cavity 4 or the electrically conductive material 2 can have the spatial shape of a parallelepiped, for example. In further examples, the cavity 4 can have a different shape, for example the shape of a cube, cylinder, sphere, etc.

The gas sensor device 200 in FIG. 2 can have some or all of the features of the gas sensor device 100 from FIG. 1. FIG. 2 shows in particular one possible more detailed implementation of a radio-frequency component 8 that can be used in a gas sensor device in accordance with the disclosure. The radio-frequency component 8 in FIG. 2 can be a wafer level package, in particular a fan-in package.

The radio-frequency component 8 can comprise a radio-frequency chip 10 and an electrical redistribution layer (or redistribution wiring layer) 14 arranged on the underside of the chip. The redistribution layer 14 can contain one or more structured metallization layers and also one or more structured dielectric layers, which can extend substantially parallel to the underside of the radio-frequency chip 10. The metal layers of the redistribution layer 14 can fulfil the function of redistribution or redistribution wiring and can be configured to provide connections of the radio-frequency chip 10 at other positions of the radio-frequency component 8. At least one radio-frequency antenna 12 can be arranged on the top side of the redistribution layer 14. In one example, the radio-frequency antenna 12 can be formed by a structured section of one or more of the metallization layers of the redistribution layer 14.

The radio-frequency component 8 can comprise contact elements 16 configured to electrically and mechanically couple the radio-frequency component 8 to a printed circuit board 18. In FIG. 2, the contact elements 16 can be embodied as solder balls, by way of example. The electrical redistribution layer 14 can provide an electrical connection between connections of the radio-frequency chip 10 and the contact elements 16.

The radio-frequency component 8 can comprise a depression 20 formed in the top side of the radio-frequency chip 10, which depression can be arranged over the radio-frequency antenna 12 and can overlap the latter as viewed in the z-direction. A path distance that has to be traversed by transmission and reception signals through the semiconductor material of the radio-frequency chip 10 can be shortened by the depression 20 formed in the top side of the radio-frequency chip 10. Power losses of the transmitted and received signals can be reduced by this means.

In the example in FIG. 2, the radio-frequency component 8 and the cavity 4 can be arranged over the top side of the printed circuit board 18. In this case, the radio-frequency component 8 can be arranged in particular inside the cavity 4. The electrically conductive material 2 forming the cavity 4 can be for example a metal cover having openings 4 formed therein. In the example in FIG. 2, the cavity 4 can be delimited by the metal cover, by the printed circuit board 18 and by the radio-frequency component 8.

In order to form a Faraday cage around the cavity 4 with improved shielding properties, electrically conductive materials 22A and 22B can be arranged on the top sides of the printed circuit board 18 and the radio-frequency component 8, respectively. By way of example, the electrically conductive materials 22A and 22B can be metal coatings. Furthermore, the radio-frequency component 8 can be shielded at its sides by one or more electrically conductive structures 24, which can be produced from a metal or a metal alloy, for example. In the side view in FIG. 2, the structure 24 can be arranged on the left and right next to the radio-frequency component 8. As viewed in the z-direction, the structure 24 can at least partly, in particular completely, surround the radio-frequency component 8. In this case, as viewed in the z-direction, the structure 24 can have an arbitrary shape, for example circular, elliptic, rectangular, square, polygonal, etc. The top side of the radio-frequency component 8 can optionally be earthed using an electrical connection between the electrically conductive structure 24 and the electrically conductive material 22B. In FIG. 2, the electrical connection can be provided by one or more wires 26 by way of example.

The gas sensor device 300 in FIG. 3 can have some or all of the features of the gas sensor device 200 from FIG. 2. Furthermore, the gas sensor device 300 can comprise one or more shielding structures 28 configured to reduce an absorption of radio-frequency signals by the at least one radio-frequency antenna 12. The shielding structure 28 can be produced from a metal or a metal alloy, for example. The shielding structure 28 can be arranged on the top side of the radio-frequency component 8 and can have the shape of an inverted funnel by way of example in the side view in FIG. 3. As viewed in the z-direction, the shielding structure 28 can at least partly, in particular completely, surround the radio-frequency antenna 12. In this case, as viewed in the z-direction, the shielding structure 28 can have an arbitrary shape, for example circular, elliptic, rectangular, square, polygonal, etc. The use of the shielding structure 28 makes it possible to increase a quality factor (in particular the conductive quality factor Q_{c}) of the cavity or cavity resonator 4 and to improve the quality of gas absorption spectra generated by the gas sensor device 300.

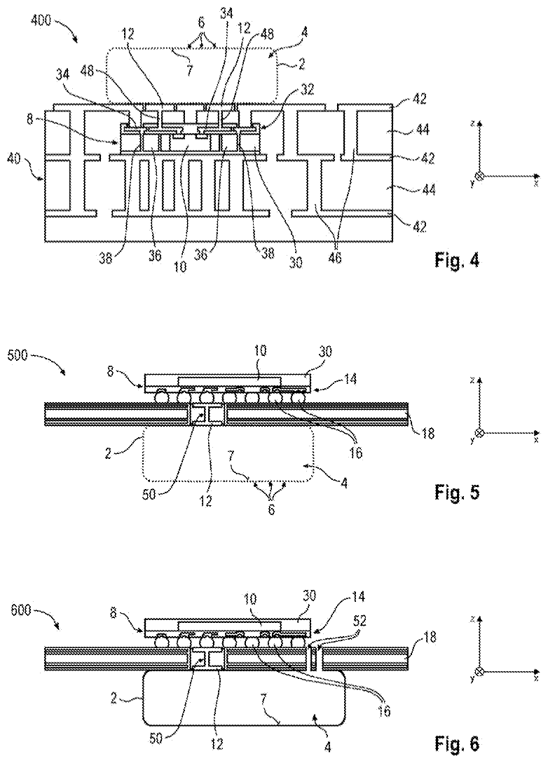

The gas sensor device 400 in FIG. 4 can have some or all of the features of the gas sensor device 100 from FIG. 1. FIG. 4 shows in particular one possible more detailed implementation of a radio-frequency component 8 that can be used in a gas sensor device in accordance with the disclosure. The radio-frequency component 8 in FIG. 4 can be a “fan-out” package, which can be produced in accordance with an eWLB (embedded Wafer Level Ball Grid Array) method.

The radio-frequency component 8 in FIG. 4 can comprise an encapsulation material 30 and a radio-frequency chip 10 embedded into the encapsulation material 30. An electrical redistribution layer (or redistribution wiring layer) 32 can be arranged over the top side of the radio-frequency chip 10 and the encapsulation material 30. The redistribution layer 32 can comprise one or more metal layers or metal tracks 34, which can extend substantially parallel to the top sides of the radio-frequency chip 10 and/or of the encapsulation material 30. The metal layers 34 of the redistribution layer 32 can fulfil the function of redistribution or redistribution wiring and can be configured to make connections of the radio-frequency chip 10 available at other locations of the arrangement. At least one radio-frequency antenna 12 can be arranged over the top side of the redistribution layer 32. FIG. 4 shows by way of example two radio-frequency antennas 12, which can be for example a transmitting antenna and a receiving antenna. The radio-frequency antennas 12 can be coupled to the radio-frequency chip 10 by way of the redistribution layer 32 and further electrical connections 48.

The radio-frequency component 8 can comprise one or more microwave components 36 having an electrically conductive wall structure 38. The respective microwave component 36 can be arranged below the respective radio-frequency antenna 12 and can be embedded into the encapsulation material 30. As viewed in the z-direction, a microwave component 36 and the radio-frequency antenna 12 arranged thereover can at least partly overlap. The electrically conductive wall structure 38 can form in particular side walls of the microwave component 36. As viewed in the z-direction, the electrically conductive wall structure 38 can thus at least partly, and in particular completely, enclose the inner region of the microwave component 36. In other words, the electrically conductive wall structure 38 can form an electrically conductive cage around the inner region of the microwave component 36 and below the radio-frequency antenna 12. In addition, the electrically conductive wall structure 38 can form a base surface of the microwave component 36. In one example, the electrically conductive wall structure 38 can comprise a multiplicity of metallized via holes. The via holes can be formed directly in the encapsulation material 30, in particular in a fan-out region of the encapsulation material 30 of the eWLB package. By way of example, the via holes can be produced in the encapsulation material 30 by way of laser drilling, and a metallization of the inner walls of the via holes can be produced using a conductive paste or metal plating, for example. The metallized via holes can be configured for heat dissipation to a metallization situated opposite the redistribution layer 32 on the underside of the radio-frequency component 8.

The microwave component 36 can be an electromagnetic shield or act as such. The radio-frequency antenna 12 can be configured inter alia to emit signals in the positive z-direction. In the case of such emission, the radio-frequency antenna 12 can also emit portions of electromagnetic radiation in the negative z-direction and also in the x- and y-directions. The microwave component 36 can be configured in particular to electromagnetically shield such signal portions emanating from one specific radio-frequency antenna 12 with respect to other radio-frequency antennas of the radio-frequency component 8. Improved isolation or separation of the transmitting and/or receiving channels provided by the radio-frequency antennas 12 of the radio-frequency component 8 can be provided by this means.

The gas sensor device 400 can comprise a printed circuit board 40, which can contain one or more metal layers or metal tracks 42, which can extend substantially in the x-direction or in the x-y-plane. The metal layers 42 can be arranged within the printed circuit board 40 and also on the top side and/or the underside of the printed circuit board 40. The metal layers 42 arranged on the top side and/or underside of the printed circuit board 40 can form contact pads of the printed circuit board 40, on which electronic components can be mounted. One or more dielectric layers 44 can be arranged between the metal layers 42 in order to electrically insulate the metal layers 42 from one another. The dielectric layers 44 can be produced for example from a PCB material, such as a fiber-reinforced plastic, in particular a composite material composed of epoxy resin and glass fiber fabric (e.g., FR4). The metal layers 42 arranged on different planes can be electrically connected to one another by a multiplicity of through contacts 46.

The metal layers 42 of the printed circuit board 40 can fulfil the function of electrical redistribution or redistribution wiring. In this case, redistribution can be provided within the printed circuit board 40 and/or between the electrical contact pads arranged on the outer sides of the printed circuit board 40. The radio-frequency component 8 can be at least partly embedded in the printed circuit board 40 or encapsulated by the latter. In this case, in particular, all surfaces of the radio-frequency component 8 can be covered by the layers of the printed circuit board 40. An electrical contacting of the radio-frequency chip 10 from outside the printed circuit board 40 can be provided by way of the metal layers 42, the through contacts 46 and the electrical redistribution layer 32 of the radio-frequency component 8.

An electrically conductive material 2 having openings 4, for example in the form of a metal cover having holes, can be arranged over the top side of the printed circuit board 40. The electrically conductive material 2 and metal layers 42 arranged on the upper main surface of the printed circuit board 40 can form a cavity or cavity resonator 4. In the example in FIG. 4, the radio-frequency component 8 can be completely embedded into the printed circuit board 40 and thus be arranged outside the cavity 4. It is thereby possible to prevent radio-frequency signals emitted into the cavity 4 and reflected multiple times at the inner surfaces 7 of the cavity 4 from being undesirably absorbed by the radio-frequency component 8. In comparison with the gas sensor devices 200 and 300 in FIGS. 2 and 3, the quality of absorption spectra generated by the gas sensor device 400 can therefore be improved.

The gas sensor device 500 in FIG. 5 can have some or all of the features of the gas sensor device 100 from FIG. 1. FIG. 5 shows one possible more detailed implementation of a radio-frequency component 8 that can be used in a gas sensor device in accordance with the disclosure. The radio-frequency component 8 in FIG. 5 can be a “fan-out” package, which can be produced in accordance with an eWLB (embedded Wafer Level Ball Grid Array) method.

The radio-frequency component 8 in FIG. 5 can comprise a radio-frequency chip 10 encapsulated in an encapsulation material 30. A redistribution layer 14 having metal layers and metal tracks can be arranged over the undersides of the radio-frequency chip 10 and of the encapsulation material 30. The radio-frequency component 8 can be mechanically and electrically coupled to a printed circuit board 18 by way of contact elements 16. The constituent parts of the radio-frequency component 8 can be similar to corresponding elements of devices that have already been described above, and so reference can be made to the preceding description for the sake of simplicity.

The radio-frequency component 8 can be arranged on the upper main surface of the printed circuit board 18. The printed circuit board 18 can comprise one or more electrically conductive structures 50, which can extend from the top side of the printed circuit board 18 to the underside of the printed circuit board 18. At least one radio-frequency antenna 12 can be arranged on the underside of the printed circuit board 18, and can be coupled to the radio-frequency chip 10 by way of the electrically conductive structure 50, by way of the contact elements 16 and by way of the electrical redistribution layer 14. An electrically conductive material 2 having openings 6 can be arranged on the lower main surface of the printed circuit board 18, the material forming a cavity 4. The radio-frequency antenna 12 can be configured to emit radio-frequency signals into the cavity 4 and to receive radio-frequency signals from the cavity 4. In the example in FIG. 5, the cavity 4 can be formed by the electrically conductive material 2 and a section of the printed circuit board 18 or metal layers arranged on the underside of the printed circuit board 18.

The radio-frequency component 8 and the cavity 4 can be arranged on opposite main surfaces of the printed circuit board 18. The radio-frequency component 8 can thus be arranged outside the cavity 4. Such an arrangement makes it possible to prevent radio-frequency signals emitted into the cavity 4 and reflected multiple times at the inner surfaces 7 of the cavity 4 from being undesirably absorbed by the radio-frequency component 8. The quality of absorption spectra generated by the gas sensor device 500 can be improved by this means.

The gas sensor device 600 in FIG. 6 can have some or all of the features of the gas sensor device 500 from FIG. 5. In contrast to FIG. 5, openings need not necessarily be formed in the electrically conductive material 2 of the gas sensor device 600. As an alternative or in addition to openings in the electrically conductive material 2, in the example in FIG. 6, one or more openings 52 can be formed in the printed circuit board 18 and enable a gas that is to be examined to penetrate into the cavity 4. In FIG. 6, by way of example, the openings 52 can be formed on the right next to the radio-frequency component 8 in the printed circuit board 18. Alternatively or additionally, further openings can be arranged on the left next to the radio-frequency component 8 and/or below the radio-frequency component 8.

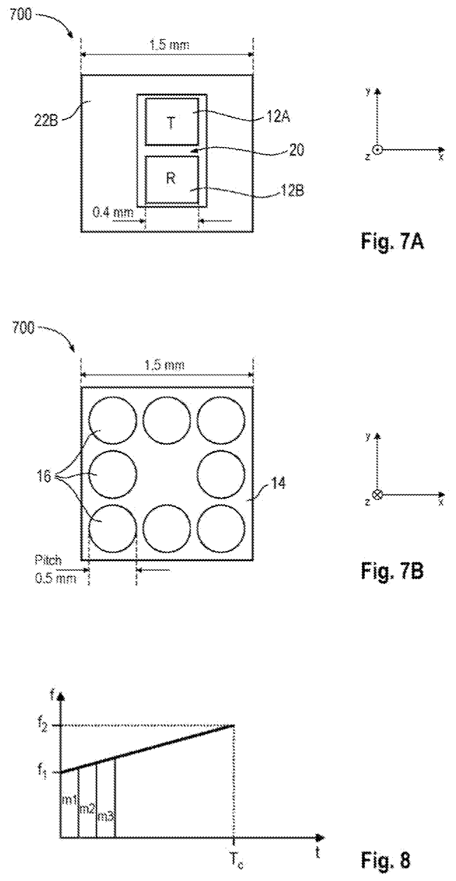

FIG. 7A shows a schematic top view of a radio-frequency component 700, while FIG. 7B illustrates a schematic bottom view of the radio-frequency component 700. The radio-frequency component 700 in FIGS. 7A and 7B can have for example some or all of the features of the radio-frequency component 8 from FIG. 2.

The top view in FIG. 7A shows an electrically conductive material 22B arranged on the top side of the radio-frequency component 700 and a depression 20 formed in the top side. A radio-frequency transmitting antenna 12A and a radio-frequency receiving antenna 12B can be arranged in the depression 20. In the top view in FIG. 7A, the radio-frequency component 700 can have by way of example a substantially square shape having an example side length in a range of approximately 0.75 mm to approximately 2.25 mm, for example approximately 1.5 mm. Furthermore, the depression 20 can have by way of example a substantially rectangular shape having an example side length in the x-direction in a range of approximately 0.2 mm to approximately 0.6 mm, for example approximately 0.4 mm.

The bottom view in FIG. 7B shows contact elements 16 arranged on the underside of the radio-frequency component 700, which contact elements can be arranged in a square pattern by way of example in FIG. 7B. By way of example, a pitch of the contact elements 16 can be in a range of approximately 0.25 mm to approximately 0.75 mm, for example approximately 0.5 mm.

Absorption spectra of a gas situated in the cavity 4 can be generated using the above-described gas sensor devices in accordance with the disclosure. In order to generate an absorption spectrum of a gas for a specific frequency range, radio-frequency signals having frequencies in this frequency range can be emitted into the cavity 4. In particular, radio-frequency signals having frequencies that change over time can be used in this case. FIG. 8 shows an example frequency profile of a chirp signal such as can be emitted into a cavity by a radio-frequency antenna in accordance with the disclosure. The frequencies of the chirp signal are plotted against time in the diagram in FIG. 8.

In the example in FIG. 8, the frequencies of the chirp signal can follow the profile of a frequency ramp between an initial frequency f₁ and a final frequency f₂. If the intention is to generate for example an absorption spectrum of a gas for a frequency range of 250 GHz to 300 GHz, a value of 250 GHz can be chosen for the initial frequency f₁ and a value of 300 GHz can be chosen for the final frequency f₂. In one specific example, the chirp signal can have a total duration T_{c} of approximately 50 μs, with the result that a gradient of approximately 1 GHz/1 μs can arise for the frequency ramp.

In FIG. 8, the total duration of the chirp signal can be divided by way of example into time intervals m₁, m₂, m₃, etc. In particular, each of the time intervals mᵢ can have a substantially identical duration, for example a duration of approximately 100 ns. At the beginning of each time interval a transmitting antenna can emit radio-frequency signals into a cavity with a frequency of the frequency ramp that is assigned to the respective point in time. The transmitted radio-frequency signal can be reflected multiple times at the inner surfaces of the cavity and can pass multiple times through the gas to be examined. At the end of the respective time interval the radio-frequency signal that has been at least partly absorbed by the gas can be received by a receiving antenna.

In one example, the receiving antenna can receive the radio-frequency signals at the end of the respective time interval mᵢ during a reception time window with an example duration of approximately 30 ns. That is to say that the reception time window assigned to the time interval m₁ of 0 ns to 100 ns, at the end of the time internal m₁, can last from approximately 70 ns to approximately 100 ns, the reception time window assigned to the time interval m₂ of 100 ns to 200 ns, at the end of the time interval m₂, can last from approximately 170 ns to approximately 200 ns, the time window assigned to the time interval m₃ of 200 ns to 300 ns, at the end of the time interval m₃, can last from approximately 270 ns to approximately 300 ns, etc. A resolution of an absorption spectrum to be generated can be increased by choosing shorter time intervals mᵢ.

Each of the gas sensor devices in accordance with the disclosure as described herein can comprise one or more switches or switching devices configured to change a terminating impedance of the at least one radio-frequency antenna of the respective gas sensor device during specific time periods. A suitable change of the terminating impedance makes it possible to reduce an absorption of radio-frequency signals by the respective radio-frequency antenna and to increase a quality of the cavity resonator. By way of example, a terminating impedance of one or more transmitting antennas can be changed between the transmission of successive radio-frequency signals. Alternatively or additionally, a terminating impedance of one or more receiving antennas can be changed between the reception of successive radio-frequency signals.

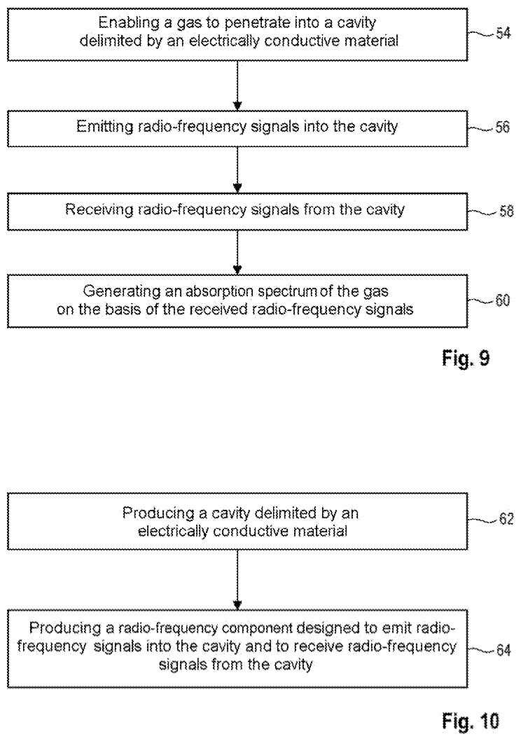

FIG. 9 shows a flow diagram of a method in accordance with the disclosure for generating an absorption spectrum of a gas. The method can be carried out for example by one of the gas sensor devices described in the preceding figures and can thus be read in association with the respective figure. The method is illustrated generally in order to qualitatively describe aspects of the disclosure, and can have further aspects. By way of example, the method can be extended by one or more of the aspects described in association with the preceding figures.

At 54, a gas can be enabled to penetrate into a cavity delimited by an electrically conductive material and having reflective surfaces by way of gas-permeable openings of the cavity. At 56, radio-frequency signals can be emitted into the cavity, wherein the radio-frequency signals are in a frequency range which comprises at least one absorption frequency of the gas. At 58, radio-frequency signals can be received from the cavity, wherein the received radio-frequency signals have passed through the gas in the cavity. At 60, the absorption spectrum of the gas can be generated based on the received radio-frequency signals.

FIG. 10 shows a flow diagram of a method in accordance with the disclosure for producing a gas sensor device. The method can be used for example to produce one of the gas sensor devices described in the preceding figures and can thus be read in association with the respective figure. The method is illustrated generally in order to qualitatively describe aspects of the disclosure, and can have further aspects. By way of example, the method can be extended by one or more of the aspects described in association with the preceding figures.

At 62, a cavity delimited by an electrically conductive material and having gas-permeable openings and reflective surfaces can be produced. At 64, a radio-frequency component can be produced, comprising a radio-frequency chip and at least one radio-frequency antenna configured to emit radio-frequency signals into the cavity and to receive radio-frequency signals from the cavity.

### ASPECTS

Gas sensor devices, associated production methods and methods for generating an absorption spectrum of a gas are explained below based on aspects.

Aspect 1 is a gas sensor device, comprising: a cavity delimited by an electrically conductive material and having gas-permeable openings and reflective surfaces; and a radio-frequency component, comprising a radio-frequency chip and at least one radio-frequency antenna configured to emit radio-frequency signals into the cavity and to receive radio-frequency signals from the cavity.

Aspect 2 is a gas sensor device according to aspect 1, wherein: the electrically conductive material forming the cavity forms a Faraday cage, and dimensions of the openings are configured to the effect that the cavity forms a cavity resonator for the radio-frequency signals emitted into the cavity.

Aspect 3 is a gas sensor device according to aspect 1 or 2, wherein: the cavity is configured to receive a gas by way of the gas-permeable openings, and the at least one radio-frequency antenna is configured to emit radio-frequency signals into the cavity in a frequency range which comprises at least one absorption frequency of the gas.

Aspect 4 is a gas sensor device according to aspect 3, furthermore comprising: a component configured to process signals received from the cavity by the at least one radio-frequency antenna and to provide an absorption spectrum of the gas based on the processed signals.

Aspect 5 is a gas sensor device according to any of the preceding aspects, wherein a dimension of the openings is smaller than a wavelength of the radio-frequency signals emitted into the cavity by the at least one radio-frequency antenna.

Aspect 6 is a gas sensor device according to any of the preceding aspects, wherein a maximum dimension of the cavity is in a range of from 2 times a wavelength of the radio-frequency signals emitted into the cavity by the at least one radio-frequency antenna up to 50 times the wavelength.

Aspect 7 is a gas sensor device according to any of the preceding aspects, wherein a frequency of the radio-frequency signals emitted into the cavity by the at least one radio-frequency antenna is in a range of 100 GHz to 1 THz.

Aspect 8 is a gas sensor device according to any of the preceding aspects, wherein the electrically conductive material comprises a metal cover.

Aspect 9 is a gas sensor device according to aspect 8, wherein the openings are formed in the metal cover.

Aspect 10 is a gas sensor device according to any of the preceding aspects, wherein the radio-frequency component is arranged inside the cavity.

Aspect 11 is a gas sensor device according to any of aspects 1 to 9, wherein the radio-frequency component is arranged outside the cavity.

Aspect 12 is a gas sensor device according to aspect 11, wherein the cavity is arranged on a main surface of a printed circuit board and the radio-frequency component is arranged on a main surface of the printed circuit board that is situated opposite the aforethe main surface.

Aspect 13 is a gas sensor device according to aspect 11, wherein the cavity is arranged on a main surface of a printed circuit board and the radio-frequency component is embedded into the printed circuit board.

Aspect 14 is a gas sensor device according to any of the preceding aspects, wherein the cavity is at least partly delimited by a printed circuit board and a part of the electrically conductive material delimiting the cavity is arranged on the printed circuit board.

Aspect 15 is a gas sensor device according to aspect 14, wherein the openings are formed in the printed circuit board.

Aspect 16 is a gas sensor device according to any of the preceding aspects, wherein the cavity is at least partly delimited by the radio-frequency component and a part of the electrically conductive material delimiting the cavity is arranged on the radio-frequency component.

Aspect 17 is a gas sensor device according to any of the preceding aspects, wherein the electrically conductive material comprises a coating arranged on an inner surface of the cavity.

Aspect 18 is a gas sensor device according to any of the preceding aspects, wherein the electrically conductive material comprises a layer stack arranged on an inner surface of the cavity, wherein the layer stack comprises at least one ferromagnetic layer and at least one electrically conductive layer.

Aspect 19 is a gas sensor device according to any of aspects 2 to 18, wherein a quality factor of the cavity resonator is greater than 10³.

Aspect 20 is a gas sensor device according to any of the preceding aspects, wherein the radio-frequency component comprises a shielding structure configured to reduce an absorption of radio-frequency signals by the at least one radio-frequency antenna.

Aspect 21 is a gas sensor device according to any of the preceding aspects, furthermore comprising: a switch configured to change a terminating impedance of the at least one radio-frequency antenna during at least one out of a time period between the emission of successive radio-frequency signals or a time period between the reception of successive radio-frequency signals.

Aspect 22 is a gas sensor device according to any of the preceding aspects, wherein the at least one radio-frequency antenna is configured to emit radio-frequency signals in the form of chirp signals.

Aspect 23 is a method for generating an absorption spectrum of a gas, wherein the method comprises: enabling a gas to penetrate into a cavity delimited by an electrically conductive material and having reflective surfaces by way of gas-permeable openings of the cavity; emitting radio-frequency signals into the cavity, wherein the radio-frequency signals are in a frequency range which comprises at least one absorption frequency of the gas; receiving radio-frequency signals from the cavity, wherein the received radio-frequency signals have passed through the gas in the cavity; and generating the absorption spectrum of the gas based on the received radio-frequency signals.

Aspect 24 is a method for producing a gas sensor device, wherein the method comprises: producing a cavity delimited by an electrically conductive material and having gas-permeable openings and reflective surfaces; and producing a radio-frequency component, comprising a radio-frequency chip and at least one radio-frequency antenna configured to emit radio-frequency signals into the cavity and to receive radio-frequency signals from the cavity.

Within the meaning of the present description, the terms “connected”, “coupled”, “electrically connected” and/or “electrically coupled” need not necessarily mean that components must be directly connected or coupled to one another. Intervening components can be present between the “connected”, “coupled”, “electrically connected” or “electrically coupled” components.

Furthermore, the words “over” and “on” used for example with respect to a material layer that is formed “over” or “on” a surface of an object or is situated “over” or “on” the surface can be used in the present description in the sense that the material layer is arranged (for example formed, deposited, etc.) “directly on”, for example in direct contact with, the surface meant. The words “over” and “on” used for example with respect to a material layer that is formed or arranged “over” or “on” a surface can also be used in the present text in the sense that the material layer is arranged (e.g., formed, deposited, etc.) “indirectly on” the surface meant, wherein for example one or more additional layers are situated between the surface meant and the material layer.

Insofar as the terms “have”, “contain”, “encompass”, “with” or variants thereof are used either in the detailed description or in the claims, these terms are intended to be inclusive in a similar manner to the term “comprise”. That means that within the meaning of the present description the terms “have”, “contain”, “encompass”, “with”, “comprise” and the like are open terms which indicate the presence of stated elements or features but do not exclude further elements or features. The articles “a/an” or “the” should be understood such that they include the plural meaning and also the singular meaning, unless the context clearly suggests a different understanding.

Furthermore, the word “example” is used in the present text in the sense that it serves as an example, a case or an illustration. An aspect or a configuration that is described as “example” in the present text should not necessarily be understood in the sense as though it has advantages over other aspects or configurations. Rather, the use of the word “example” is intended to present concepts in a concrete manner. Within the meaning of this application, the term “or” does not mean an exclusive “or”, but rather an inclusive “or”. That is to say that, unless indicated otherwise or unless a different interpretation is allowed by the context, “X uses A or B” means each of the natural inclusive permutations. That is to say if X uses A, X uses B or X uses both A and B, then “X uses A or B” is fulfilled in each of the cases mentioned above. Moreover, the articles “a/an” can be interpreted within the meaning of this application and the accompanying claims generally as “one or more”, unless it is expressly stated or clearly evident from the context that only a singular is meant. Furthermore, at least one out of A or B or the like generally means A or B or both A and B.

Devices and methods for producing devices are described in the present description. Observations made in connection with a device described can also apply to a corresponding method, and vice versa. If a specific component of a device is described, for example, then a corresponding method for producing the device can contain an action for providing the component in a suitable manner, even if such an action is not explicitly described or illustrated in the figures. Moreover, the features of the various example aspects described in the present text can be combined with one another, unless expressly noted otherwise.

Although the disclosure has been shown and described with respect to one or more implementations, equivalent alterations and modifications based at least in part on the reading and understanding of this description and the accompanying drawings will be apparent to the person skilled in the art. The disclosure includes all such modifications and alterations and is restricted solely by the concept of the following claims. Especially with respect to the various functions that are implemented by the above-described components (for example elements, resources, etc.), the intention is that, unless indicated otherwise, the terms used for describing such components correspond to any components which implement the specified function of the described component (which is functionally equivalent, for example), even if it is not structurally equivalent to the disclosed structure which implements the function of the example implementations of the disclosure as presented herein. Furthermore, even if a specific feature of the disclosure has been disclosed with respect to only one of various implementations, such a feature can be combined with one or more other features of the other implementations in a manner such as is desired and advantageous for a given or specific application.

1. A gas sensor device, comprising:
an electrically conductive material that defines a cavity, wherein the electrically conductive material includes gas-permeable openings and reflective surfaces; and
a radio-frequency component comprising a radio-frequency chip and at least one radio-frequency antenna configured to emit radio-frequency signals into the cavity and to receive reflected radio-frequency signals from the cavity, wherein the reflected radio-frequency signals correspond to the radio-frequency signals reflected by the reflective surfaces.

2. The gas sensor device according to claim 1, wherein:
the electrically conductive material forming the cavity forms a Faraday cage, and
dimensions of the gas-permeable openings are configured to an effect that the cavity forms a cavity resonator for the radio-frequency signals emitted into the cavity.

3. The gas sensor device according to claim 1, wherein:
the cavity is configured to receive a gas by way of the gas-permeable openings, and
the at least one radio-frequency antenna is configured to emit the radio-frequency signals into the cavity in a frequency range which comprises at least one absorption frequency of the gas.

4. The gas sensor device according to claim 3, further comprising:
a processing component configured to process the reflected radio-frequency signals received from the cavity by the at least one radio-frequency antenna and to provide an absorption spectrum of the gas based on the reflected radio-frequency signals.

5. The gas sensor device according to claim 1, wherein a dimension of each of the gas-permeable openings is smaller than a wavelength of the radio-frequency signals emitted into the cavity by the at least one radio-frequency antenna.

6. The gas sensor device according to claim 1, wherein a maximum dimension of the cavity is in a range of from 2 times a wavelength of the radio-frequency signals emitted into the cavity by the at least one radio-frequency antenna up to 50 times the wavelength.

7. The gas sensor device according to claim 1, wherein a frequency of the radio-frequency signals emitted into the cavity by the at least one radio-frequency antenna is in a range of 100 GHz to 1 THz.

8. The gas sensor device according to claim 1, wherein the electrically conductive material comprises a metal cover.

9. The gas sensor device according to claim 8, wherein the gas-permeable openings are formed in the metal cover.

10. The gas sensor device according to claim 1, wherein the radio-frequency component is arranged inside the cavity.

11. The gas sensor device according to claim 1, wherein the radio-frequency component is arranged outside the cavity.

12. The gas sensor device according to claim 11, wherein the cavity is arranged on a first main surface of a printed circuit board and the radio-frequency component is arranged on a second main surface of the printed circuit board that is situated opposite to the first main surface.

13. The gas sensor device according to claim 11, wherein the cavity is arranged on a main surface of a printed circuit board and the radio-frequency component is embedded into the printed circuit board.

14. The gas sensor device according to claim 1, wherein the cavity is at least partly delimited by a printed circuit board and a part of the electrically conductive material delimiting the cavity is arranged on the printed circuit board.

15. The gas sensor device according to claim 14, wherein the gas-permeable openings are formed in the printed circuit board.

16. The gas sensor device according to claim 1, wherein the cavity is at least partly delimited by the radio-frequency component and a part of the electrically conductive material delimiting the cavity is arranged on the radio-frequency component.

17. The gas sensor device according to claim 1, wherein the electrically conductive material comprises a coating arranged on an inner surface of the electrically conductive material.

18. The gas sensor device according to claim 1, wherein the electrically conductive material comprises a layer stack arranged on an inner surface of the electrically conductive material that defines a boundary of the cavity, wherein the layer stack comprises at least one ferromagnetic layer and at least one electrically conductive layer.

19. The gas sensor device according to claim 2, wherein a quality factor of the cavity resonator is greater than 10³.

20. The gas sensor device according to claim 1, wherein the radio-frequency component comprises a shielding structure configured to reduce an absorption of one or more radio-frequency signals by the at least one radio-frequency antenna.

21. The gas sensor device according to claim 1, further comprising:
a switch configured to change a terminating impedance of the at least one radio-frequency antenna during at least one of a time period between an emission of successive radio-frequency signals or a time period between a reception of successive reflected radio-frequency signals.

22. The gas sensor device according to claim 1, wherein the at least one radio-frequency antenna is configured to emit the radio-frequency signals in the form of chirp signals.

23. A method for generating an absorption spectrum of a gas, wherein the method comprises:
enabling a gas to penetrate into a cavity delimited by an electrically conductive material having inner reflective surfaces, wherein the gas penetrates into the cavity by way of gas-permeable openings of the electrically conductive material;
emitting radio-frequency signals into the cavity via the gas-permeable openings, wherein the radio-frequency signals are in a frequency range which comprises at least one absorption frequency of the gas;
receiving reflected radio-frequency signals from the cavity, wherein the reflected radio-frequency signals have passed through the gas inside the cavity and have been reflected by the inner reflective surfaces; and
generating the absorption spectrum of the gas based on the reflected radio-frequency signals.

24. A method for producing a gas sensor device, wherein the method comprises:
producing a cavity delimited by an electrically conductive material having gas-permeable openings and inner reflective surfaces; and
producing a radio-frequency component, comprising a radio-frequency chip and at least one radio-frequency antenna configured to emit radio-frequency signals into the cavity via the gas-permeable openings and receive reflected radio-frequency signals from the cavity, the reflected radio-frequency signals correspond to the radio-frequency signals reflected by the inner reflective surfaces.
